(19) United States
(12) Patent Application Publication
(10) Pub. No.: US 2016/0175412 A1
(43) Pub. Date: Jun. 23, 2016

(54) PREVENTION OF TYPE 1 DIABETES BY TREG VACCINATION WITH AN INSULIN MIMETOPE

(71) Applicant: DANA-FARBER CANCER INSTITUTE, INC., BOSTON, MA (US)

(72) Inventors: HARALD VON BOEHMER, BROOKLINE, MA (US); CAROLIN DANIEL, BROOKLINE, MA (US)

(21) Appl. No.: 14/834,665

(22) Filed: Aug. 25, 2015

Related U.S. Application Data
(63) Continuation of application No. 13/157,940, filed on Jun. 10, 2011, now abandoned.
(60) Provisional application No. 61/354,107, filed on Jun. 11, 2010.

(51) Int. Cl. A61K 39/00; A61K 47/48

(52) U.S. Cl. CPC A61K 39/0005; A61K 47/48415; A61K 2039/57; A61K 2039/6056

(57) ABSTRACT

The invention involves methods and products for inducing Treg cells for immune suppression in connection with autoimmune disease and transplant rejection.

### RELATED APPLICATION

This application claims priority under 35 U.S.C. §119 from U.S. provisional application Ser. No. 61/354,107 filed Jun. 11, 2010, the entire content of which is incorporated herein in its entirety.

### GOVERNMENT SUPPORT

This invention was made with Government support under NIH grant R37AI0531102-08. Accordingly, the Government has certain rights in the invention.

### BACKGROUND OF INVENTION

Several mechanisms account for self-nonself discrimination by the immune system: Clonal deletion of autoreactive T cells within the thymus represents one important mechanism. Despite its high efficiency, it is insufficient to prevent the accumulation of self-reactive T cells in peripheral lymphoid tissues. These escapees can be kept in check by CD4⁺ CD25⁺ Foxp3-expressing regulatory T cells (Tregs), which suppress their activation and effector function (1-3). Importantly, upon antigenic stimulation, Tregs can control responses of neighboring T cells with different antigen specificity in vivo (2, 4, 5).

A variety of experiments indicated that Tregs can delay the onset or cure mice of diabetes, allergy, colitis, and graft-versus-host disease (GVHD) or interfere with graft rejection (6-14). Some of these experiments indicate that the efficacy of Treg-based immune therapy crucially depends on the antigen specificity of Tregs. On the other hand, one of the major limitations to devise strategies for the clinical use of Tregs is the difficulty in obtaining antigen-specific Tregs.

Antigen-specific Foxp3⁺ Tregs can be induced by either expressing antigens in certain tissue, such as thymic epithelial cells, or delivering antigen under sub-immunogenic conditions (15-18). In T cell antigen receptor (TCR)-transgenic mice, foreign TCR agonist peptides, when supplied over a 2-week period through infusion by implanted osmotic minipumps or by antigen-DEC205 fusion antibodies targeting dendritic cells, could be used to instruct naive T cells to become Foxp3⁺ Tregs, which mediated tolerance and could persist for long periods of time in the absence of their inducing antigen (17, 18).

The de novo induction of antigen-specific Foxp3⁺ Tregs has been documented unambiguously with T cells with transgenic TCRs specific for a limited number of antigens (17, 18), consistent with earlier work that were suggestive of Treg mediated tolerance under similar experimental conditions (19-21). It is important to establish that lessons learned from artificial transgenic systems can be translated to nontransgenic organisms. Such a task requires visualization of antigen-specific Tregs in WT mice, where the very low frequency of T cells with a given antigen specificity makes it difficult especially because antigen-induced conversion takes place mostly in nondividing cells (17, 18). However, such converted Treg can be expanded by antigenic stimulation.

The generation of HY-specific Tregs which interfere with HY antigen-specific graft rejection has also been described. Graft rejection represents a serious complication in human transplantation (22) and can be conveniently studied in mice. In H-2^{b} mice, the HY-specific response is largely restricted to single immunodominant class II and class I MHC-presented epitopes (22, 23).

Because the supply of antigen by implanted osmotic minipumps is effective in inducing Tregs over a wide dose range of peptides, whereas delivery via DEC205 fusion antibodies works in a relatively narrow dose range, the experiments were done using osmotic minipumps for convenience. The impact of peptide infusion on both CD4 and CD8 T cell responses was directly analyzed by visualizing male-specific CD8 and CD4 T cells with the aid of MHC class I and class II HY-specific peptide tetramers. The results showed that the supply of peptide under sub-immunogenic conditions can induce complete transplantation tolerance in WT mice by converting naïve HY-specific CD4⁺ T cells into Foxp3⁺ Tregs, which in turn suppress the response of male-specific CD4 and CD8 T cells even when the latter recognize peptides from a different HY protein.

### SUMMARY OF INVENTION

It has been discovered, surprisingly, that strong mimetopes of weak antigens can be more effective than the weak antigens to induce immune suppression. In particular, it has been discovered that mimetopes which are stronger activators of T cell proliferation and, therefore, more immunogenic, can be more effective in inducing antigen specific Treg cells when used at sub-immunogenic doses and in subjects lacking activated T cells specific for the antigen. It was unexpected, and counter intuitive, that a strong T cell antigen would be more effective to induce immune suppression than a weak T cell antigen.

Thus, the invention involves in some aspects identifying weak native antigens characteristic of autoimmune disease and transplant rejection and preparing mimetopes of those weak native antigens, which mimetopes are stronger activators of T cell proliferation. Those mimetopes are then used in sub-immunogenic does to generate in vitro or in vivo Treg cells that specifically bind the native antigens. The mimetopes can be used to induce immune suppression in certain subjects in need thereof, such as in connection with reagents inhibiting the development of autoimmune disease or transplant rejection. The mimetopes in another aspect to are used in pharmaceutical preparations. In addition, novel mimetopes are provided as well as methods for identifying mimetopes useful according to the invention.

According to one aspect of the invention, a method is provided for increasing in a subject Treg cells specific for an antigen. The method involves administering to the subject, under sub-immunogenic conditions, a mimetope of the antigen, wherein the mimetope has a mimetope TCP index, and wherein the mimetope TCP index is at least 25% greater than the antigen TCP index under the same conditions of T cell proliferation. In some embodiments, the mimetope TCP index is greater, under the same conditions, as the antigen TCP index by at least 50%, 100%, 200%, 300%, 400%, 500% or 1000%. In any of the embodiments, the mimetope can be administered under sub-immunogenic conditions subcutaneously by an osmotic pump. In any of the embodiments, the mimetope can be administered under sub-immunogenic conditions by injection of a covalent conjugate of the mimetope and an antibody that binds specifically to dendritic cells.

In some embodiments, the mimetope is a peptide and the antigen is a native protein. In some embodiments, the peptide is represented by a sequence of contiguous amino acids, which sequence is present as contiguous amino acids found in the protein, except for one amino acid, and wherein the one amino acid is part of an MHC binding portion of the mimetope. In some embodiments, the peptide is represented by a sequence of contiguous amino acids, which sequence is present as contiguous amino acids found in the protein, except for two amino acids, and wherein at least one of the two amino acids is part of an MHC binding portion of the mimetope. In some embodiments, each of the two amino acids is part of the MHC binding portion of the mimetope. In some embodiments, the peptide comprises a sequence X₁, X₂, X₃, X₄, X₅, X₆, X₇, wherein each X represents an amino acid, and wherein the sequence differs from a sequence of contiguous amino acids found within the protein at only one of X₁, X₂, X₃, X₄, X₅, X₆, X₇, and wherein said only one is part of an MHC binding portion of the mimetope. In some embodiments, the peptide comprises a sequence X₁, X₂, X₃, X₄, X₅, X₆, X₇, wherein each X represents an amino acid, and wherein the sequence differs from a sequence of contiguous amino acids found within the protein at only two of X₁, X₂, X₃, X₄, X₅, X₆, X₇, and wherein said only two are part of the same MHC binding portion of the mimetope.

In some aspects of the invention, the antigen is a self antigen involved in autoimmune disease. In some embodiments, the autoimmune disease is Multiple Sclerosis, autoimmune myocarditis, pemphigus, celiac disease, myasthenia gravis, Hashimoto's thyroiditis, Graves' disease, Addison's disease, chronic lyme arthritis, Goodpasture syndrome, Kawasaki disease, scleroderma, Sjogren's syndrome.

In some embodiments, the antigen is insulin, myelin basic protein, cardiac myosin, Pemphigus vulgaris antigen (Desmoglein 3), gliadin, muscle acetylcholine receptor, ryanodine receptor 1, thyroid peroxidase, thyroglobulin, thyroid peroxidase, a sodium-iodide symporter, a thyrotropin receptor, a cytoplasmic adrenal antigen, a P450 enzyme 21-hydroxylase, a 11 beta-hydroxylase, a 17 alpha-hydroxylase, a side-chain cleavage enzyme P450, a 3 beta-hydroxysteroid dehydrogenase, a polypeptide of Borrelia Burgdorferi and its outer surface proteins, a collagen, type IV, alpha 3, a factor VIII related antigen (von Willebrand's Factor), an adenoviral antigen, a protein subunit of human RNAse P, a fibrillarin, a Lupus La protein, and Bet v 1. In some embodiments, the mimetope is a mimetope of natural insulin B:9-23 peptide, Chromogranin A or myelin basic protein. In some embodiments, the mimetope is InsMim3 peptide mimetope, InsMim8 peptide mimetope, pS3 peptide mimetope, Ac1-11 A4 MBP peptide mimetope, or Ac1-11 Y4 MBP peptide mimetope.

According to some aspects of the invention, the subject is a candidate for a transplant and the antigen is a non-self antigen present in the transplant but not in the subject. In any of these embodiments, the antigen can be a MHC antigen. In some embodiments, the mimetope is E62M peptide mimetope.

According to some aspects of the invention, the subject is a candidate for treatment to inhibit the development of IgE-mediated allergy and/or allergic symptoms and the antigen is an allergen. In some embodiments, the mimetope is a mimetope of Bet v 1.

In aspects of the invention, the subject does not have detectable cytotoxic T cells specific for the antigen. In some embodiments, the subject is treated prophylactically. In some embodiments, the subject is at an enhanced risk relative to the general population for developing disease. In some embodiments, the subject has signs of disease, but has not progressed to the disease state. In some embodiments, the subject has no detectable antibodies to the antigen. In some embodiments the disease is autoimmune disease. In some embodiments, the subject is a candidate for a heart transplant or a kidney transplant. In some embodiments the subject is prone to develop allergy.

In other aspects of the invention, a pharmaceutical preparation for inhibiting autoimmune disease is provided. The preparation is a sub-immunogenic dose of a peptide mimetope of a self antigen involved in auto-immune disease, wherein the peptide mimetope has a mimetope TCP index, and wherein the mimetope TCP index is at least 25% greater than an antigen TCP index under the same conditions of T cell proliferation. In some embodiments, the peptide mimetope has a sequence X₁, X₂, X₃, X₄, X₅, X₆, X₇, wherein each X represents an amino acid, and wherein the sequence differs from a sequence of contiguous amino acids found within the antigen at only one or two of X₁, X₂, X₃, X₄, X₅, X₆, X₇, and wherein said only one or two is part of an MHC binding portion of the peptide mimetope and the antigen.

In some embodiments, the self antigen is involved in Multiple Sclerosis, autoimmune myocarditis, pemphigus, celiac disease, myasthenia gravis, Hashimoto's thyroiditis, Graves' disease, Addison's disease, chronic lyme arthritis, Goodpasture syndrome, Kawasaki disease, scleroderma, or Sjogren's syndrome or allergy. In some embodiments, the antigen is insulin, myelin basic protein, cardiac myosin, Pemphigus vulgaris antigen (Desmoglein 3), gliadin, muscle acetylcholine receptor, ryanodine receptor 1, thyroid peroxidase, thyroglobulin, thyroid peroxidase, a sodium-iodide symporter, a thyrotropin receptor, a cytoplasmic adrenal antigen, a P450 enzyme 21-hydroxylase, a 11 beta-hydroxylase, a 17 alpha-hydroxylase, a side-chain cleavage enzyme P450, a 3 beta-hydroxysteroid dehydrogenase, a polypeptide of Borrelia Burgdorferi and its outer surface proteins, a collagen, type IV, alpha 3, a factor VIII related antigen (von Willebrand's Factor), an adenoviral antigen, a protein subunit of human RNAse P, a fibrillarin, a Lupus La protein, or a pollen allergen as for example Bet v 1—the dominant birch pollen allergen. In some embodiments, the peptide mimetope is a mimetope of natural insulin B:9-23 peptide.

In any of the forgoing embodiments, the pharmaceutical preparation can be in an osmotic pump. In any of the forgoing embodiments, the mimetope can be covalently conjugated to an antibody that binds specifically to a dendritic cell.

According to another aspect of the invention, a pharmaceutical preparation for treating transplant rejection is provided. The preparation includes a sub-immunogenic dose of a peptide to mimetope of a non-self transplant antigen, wherein the peptide mimetope has a mimetope TCP index, and wherein the mimetope TCP index is at least 25% greater than an antigen TCP index under the same conditions of T cell proliferation. In some embodiments, the peptide mimetope has a sequence X₁, X₂, X₃, X₄, X₅, X₆, X₇, wherein each X represents an amino acid, and wherein the sequence differs from a sequence of contiguous amino acids found within the antigen at only one or two of X₁, X₂, X₃, X₄, X₅, X₆, X₇, and wherein said only one or two is part of an MHC binding portion of the peptide mimetope and the antigen. In some embodiments, the antigen is an MHC antigen. In any of the foregoing embodiments, the pharmaceutical preparation can be in an osmotic pump. In any of the foregoing embodiments, the mimetope can be covalently conjugated to an antibody that binds specifically to a dendritic cell.

According to other aspects of the invention, Treg cells are prepared in vitro, for use in experimentation or for use in therapy. When used in therapy, in some embodiments, the peripheral T cells are obtained from a subject, Treg cells specific for an antigen are induced, and the Treg cells are introduced into the subject.

According to another aspect of the invention, novel peptides are provided.

These and other aspects of the invention are described in more detail below.

### BRIEF DESCRIPTION OF DRAWINGS

FIG. 1 shows the impact of certain compounds on conversion and stability of induced Tregs;

FIG. 2 shows thymidine incorporation after stimulation of NOD InsB:9-23 Tg T cells with Insulin-derived Peptides;

FIG. 3 shows an experimental scheme for in vivo conversion of CD4+CD25− InsB:9-23 TCR transgenic T cells after transfer into Thy1.1+NOD mice;

FIG. 4 shows in vivo conversion of CD4+CD25− InsB:9-23 TCR transgenic T cells into Tregs after transfer into Thy1.1+NOD mice;

FIG. 5 shows induction of tolerance to insulin-derived peptides in a model of the development of diabetes; and

FIG. 6 shows the impact of Treg-enhancing compounds on induction of tolerance to Insulin-derived peptides in a model of the development of diabetes.

### DETAILED DESCRIPTION OF INVENTION

### Definitions

“Amino acid” means any one of the twenty naturally-occurring amino acids or the D-form of any one of the naturally-occurring amino acids. In addition, the term “amino acid” also includes other non-naturally occurring amino acids besides the D-amino acids, including synthetic amino acids and modified amino acids. Examples of non-naturally-occurring amino acids include, but are not limited to, norleucine (“Nle”), norvaline (“Nva”), (3-Alanine, L- or D-naphthalanine, ornithine (“Orn”), homoarginine (homoArg) and others well known in the peptide art, such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, both of which are incorporated herein by reference Amino acids and amino acid analogs can be purchased commercially (Sigma Chemical Co.; Advanced Chemtech) or synthesized using methods known in the art.

“Mimetope” means an antigen that differs from a native antigen but nonetheless binds MHC and induces an specific immune response to that native antigen.

“Native antigen” means an antigen which is a normal part of an animal in nature or of an allergen.

“Subimmunogenic” means conditions which avoid activation of antigen presenting cells.

“Subject” means a mammal, including, but not limited to, a human, a dog, a cat, a horse, and a rodent.

“TCP index” means a measure of the ability of an antigen to activate T cells, which can be a measure of the ability of an antigen to induce T cell proliferation.

The invention involves identification and use of mimetopes of antigens, typically native antigens, wherein the mimetopes have a substantially higher TCP index than the TCP index of to the antigen. The antigens in some embodiments are those which are involved in autoimmune disease or transplant rejection.

A non-limiting list of autoimmune diseases and corresponding antigens known to be involved in autoimmune disease is as follows.

| Autoimmune disease | Antigen |
|---|---|
| Multiple sclerosis | Myelin basic protein |
| autoimmune myocarditis, | cardiac myosin, |
| pemphigus, | Pemphigus vulgaris antigen (Desmoglein 3) |
| celiac disease, | Gliadin, and relevant gliadin peptides |
| myasthenia gravis, | muscle acetylcholine receptor and relevant peptide, a |
| | 146-162, Ryanodine receptor 1 |
| Hashimoto's thyroiditis, | Thyroid peroxidase, see in addition at Graves' disease |
| Graves' disease, | thyroglobulin, thyroid peroxidase, sodium-iodide |
| | symporter, and the thyrotropin receptor |
| Addison's disease, | cytoplasmic adrenal autoantibodies, P450 enzyme 21- |
| | hydroxylase, a164-356, 11 beta-hydroxylase, 17 alpha- |
| | hydroxylase, side-chain cleavage enzyme P450, and 3 |
| | beta-hydroxysteroid dehydrogenase |
| chronic lyme arthritis, | Polypeptide of Borrelia Burgdorferi and its outer |
| | surface proteins |
| Goodpasture syndrome, | Collagen, type IV, alpha 3 |
| Kawasaki disease | Factor VIII related antigen (von Willebrand's Factor), |
| | acute adenoviral infection |
| scleroderma, | Protein subunit of human RNAse P, Fibrillarin, |
| | antibodies can be detected against extractable nuclear |
| | antigens |
| Sjogren's syndrome | Lupus La protein |

In addition to autoimmune disease, the invention is useful in connection with increasing Tregs specific for allergens, to inhibit of the development of IgE-allergy and/or allergic symptoms. Allergens are well known. One particularly relevant allergen is Bet v 1, involved with birch pollen allergy. MHC class II restricted CD4+ T cells for Bet v 1 have been identified.

Typically, the antigen for which the mimetope will be made is relatively weak as an activator of T cells. When introduced in vivo, an antigen may be processed by antigen presenting cells into small peptides, which bind to MHC (the “MHC binding portion” or “pocket” as used herein), which small peptides are presented, bound to MHC, to T Cells by the to antigen presenting cells. The MHC binding portion typically will fit 7-8 amino acids of the native antigen. Several of these amino acids will ‘contact’ corresponding points of the pocket. Native antigens that are poor activators of T cells can bind this pocket weakly.

According to the invention, mimetopes are identified which are stronger activators of T cells than their native counterparts. The mimetopes can be found in nature or identified from libraries Mimetopes also can be designed rationally, by examining the portion of an antigen that binds MHC and in what register(s) and making single (or more) amino acid changes at a particular position where a change would be predicted to improve binding. Such changes can alter the size, configuration and/or charge of the peptide and, accordingly, the strength of binding to MHC. This latter approach was taken for identifying the mimetopes for use in the invention. Preparing a mimetope of an antigen is well within the ordinary skill of the art.

The relative strength of T cell activation can be measured versus that of the native antigen (or peptide portion of the native antigen) in a variety of ways. An exemplary test used to determine and quantify the ability of insulin and insulin mimetopes to activate T cell is described briefly in the examples below and in more detail in Alkan S, Antigen-induced proliferation assay for mouse T lymphocytes. Response to a monovalent antigen, Eur J Immunol 1978, 8(2) 112-118. The methods for conducting in vivo and in vitro tests of Treg induction are likewise described in the examples below and in references cited below (e.g., 24). These references do not discuss, however, identifying mimetopes of weak antigens or the use of a mimetope of a weak antigen for inducing Treg cells, but instead provide a detailed description of the general protocols for inducing Treg cells using sub-immunogenic conditions. These same methods can be used to identify and test mimetopes to be used according to the invention.

Examples of particular mimetopes useful according to the invention are described below. These mimetopes were identified using the methods described herein, as well as based on rational predictions.

Natural Insulin B:9-23 Peptide.

The natural Insulin B:9-23 peptide is known to be associated with Type I diabetes. This peptide binds with poor affinity to IAg⁷ in multiple binding registers. Exchange in p9 from Arginine to Glutamic acid results in enhanced MHC binding and better stimulation of T cells.

Insulin B 9: 23:
(SEQ ID NO: 1)

| | | | | | | | | | | | | | | |
|---|---|---|---|---|---|---|---|---|---|---|---|---|---|---|
| S | H | L | V | E | A | L | Y | L | V | C | G | E | R | G |

InsMim3 peptide mimetope:
(SEQ ID NO: 2)

| | | | | | | | | | | | |
|---|---|---|---|---|---|---|---|---|---|---|---|
| V | E | A | L | Y | L | V | C | G | E | E | G |
| p1 | | | p4 | | p6 | | | p9 | | | |
| | p1 | | | p4 | | p6 | | | p9 | | |
| | | p1 | | | p4 | | p6 | | | p9 | |

Variations of this Insulin-Mimetope have been identified to increase MHC-binding and avoid peptide dimerization.

InsMim8 peptide mimetope:
(SEQ ID NO: 3)

| | | | | | | | | | | | |
|---|---|---|---|---|---|---|---|---|---|---|---|
| V | E | A | L | Y | L | V | A | G | E | E | G |
| p1 | | | p4 | | p6 | | | p9 | | | |
| | p1 | | | p4 | | p6 | | | p9 | | |
| | | p1 | | | p4 | | p6 | | | p9 | |

### Chromogranin A.

Chromogranin A was recently identified as the antigenic source of the highly pathogenic BDC2.5 T cell clone associated with Type I diabetes. WE14 is naturally processed from Chromogranin A by a furin-like protease. As with the natural Insulin B9:23 peptide, the WE14 binds to IAg⁷ weakly and induces poor stimulation of BDC2.5 T cells. Studies have shown that this epitope only partially fills the peptide binding groove. The pS3 peptide was identified as a strong stimulating mimetope with improved MHC-binding.

| WE14 peptide | | | | | | | | | | | | | |
|---|---|---|---|---|---|---|---|---|---|---|---|---|---|
| (SEQ ID NO: 4) | | | | | | | | | | | | | |
| W | S | R | M | D | Q | L | A | K | E | L | T | A | E |
| (SEQ ID NO: 5) | | | | | | | | | | | | | |
| pS3 peptide mimetope | | | | | | | | | | | | | |
| R | L | G | L | W | V | R | M | E | | | | | |

MHC Peptides

The natural HY-peptide is a good agonist itself and can be used to induce Treg cells in the methods described herein. Therefore, a mimetope possessing better binding and stimulatory properties is not required. For other transplantation antigens, such as the MHC-derived K^{d}₅₄₋₆₈ peptide, it is known that this peptide is a weak agonist. Dendritic cells pulsed with the K^{d}₅₄₋₆₈ peptide are not able to stimulate the proliferation of T cells with a specific transgenic TCR for the K^{d}₅₄₋₆₈ peptide. Under these conditions the identification of a mimetope including better binding and stimulatory capacities is indicated.

Kd 54-68: MHC class I derived processed peptide presented by IAb

Kd 54-68 peptide:
(SEQ ID NO: 6)

| | | | | | | | | | | | | | | |
|---|---|---|---|---|---|---|---|---|---|---|---|---|---|---|
| Q | E | G | P | E | Y | W | E | E | Q | T | Q | R | A | K |

E62M peptide mimetope:
(SEQ ID NO: 7)

| | | | | | | | | | | | | | | |
|---|---|---|---|---|---|---|---|---|---|---|---|---|---|---|
| Q | E | G | P | E | Y | W | E | M | Q | T | Q | R | A | K |

The immunodominant encephalitogenic T cell epitope of myelin basic protein (MBP) Acetylated N-terminal Act-11 MBP presented by IAu is a weak epitope.

Ac1-11 MBP peptide:
(SEQ ID NO: 8)

| | | | | | | | | | | | |
|---|---|---|---|---|---|---|---|---|---|---|---|
| Ac | A | S | Q | K | R | P | S | Q | R | H | G |

Ac1-11 A4 MBP peptide mimetope
(SEQ ID NO: 9)

| | | | | | | | | | | | |
|---|---|---|---|---|---|---|---|---|---|---|---|
| Ac | A | S | Q | A | R | P | S | Q | R | H | G |

Ac1-11 Y4 MBP peptide mimetope
(SEQ ID NO: 10)

| | | | | | | | | | | | |
|---|---|---|---|---|---|---|---|---|---|---|---|
| Ac | A | S | Q | Y | R | P | S | Q | R | H | G |

The phenomenon of ‘weak natural peptides’ seems to be common among autoimmune settings as type 1 diabetes, Multiple sclerosis, and, it is believed, other autoimmune disorders, perhaps in order to escape negative selection of autoreactive T cells in the thymus. The phenomenon of ‘weak natural peptides’ also could play a role in transplantation when the relevant transplantation antigens are weak agonists.

The pharmaceutical preparations of the invention are administered in therapeutically effective amounts. A therapeutically effective amount will be determined by the parameters discussed below; but, in any event, is that amount which establishes a level of the drug(s) effective for treating a subject, such as a human subject, having one of the conditions described to herein. An effective amount means that amount alone or with multiple doses, necessary to delay the onset of, inhibit completely or lessen the progression of or halt altogether the onset or progression of the condition being treated. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation.

The pharmaceutical preparations of the present invention may include or be diluted into a pharmaceutically-acceptable carrier. The term “pharmaceutically-acceptable carrier” as used herein means one or more compatible fillers, diluents or other such substances, which are suitable for administration to a human or other mammal such as a dog, cat, or horse. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carriers suitable for oral, subcutaneous, intravenous, intramuscular, etc. formulations can be found in Remington's Pharmaceutical Sciences, Mack Publishing Company, Easton, Pa.

Generally, subcutaneous doses of active compounds, administered by osmotic pump, will be from about 5-500 μg/kg of body weight, dosed continuously over 14 days. For the intraperitoneal anti-DEC205 application route: 0.5-2 μg/kg anti-DEC205 fusion antibody (referring to 0.05-0.2 μg/kg of antigen contained in the fusion antibody) injected intraperitoneally. In both cases, the application of the antigen should be accompanied by a Treg-enhancing drug regime interfering with costimulation to allow for maintenance of subimmunogenic conditions e.g. avoidance of activation of antigen-presenting cells (mTOR-inhibitor everolimus).

### Examples

### 1: In Vitro Assay for Assessing Native Antigen and Mimetope Activation of T Cells: Thymidine Incorporation Assay

A key feature of the adaptive immune response is the ability of antigen-specific T cells to rapidly proliferate following antigenic-stimulation. Assays to assess the proliferative activity of antigen-specific T cells are a core tool to allow for evaluation of TCR- and MHC-binding properties based on the growth rate of the T cell populations after stimulation with a defined amount of antigen. A historically established assay is the detection of tritiated thymidine ³H uptake. Therefore CD4+ T-cells from Rag def. NOD mice expressing a transgenic TCR for the a peptide of interest (e.g., Insulin B 9:23 peptide) are isolated by the use of a magnetic bead separation approach. Purified CD11⁺DCs from normal NOD mice are used as antigen-presenting cells and irradiated. To induce proliferation of antigen-specific CD4⁺ T cells, co-cultures are set up with naïve Insulin-specific CD4⁺ T cells in 200 μl RPMI1640 medium supplemented with 10% (vol/vol) FCS in 96-well round-bottomed plates. Soluble peptides (e.g., natural InsulinB9:23 peptide) or mimetopes (e.g., Insulin mimetopes) are added to the cultures in various concentration (10 μg/ml to 0.01 μg/ml). Analysis of proliferation of antigen-specific T cells is performed by incorporation of ³H-thymidine added for the last 12 hours of a 70 hours culture period followed by scintillation counting. (A more detailed example of this procedure can be found in Alkan S, Antigen-induced proliferation assay for mouse T lymphocytes. Response to a monovalent antigen, Eur J Immunol 1978, 8(2) 112-118.

### 2. Peripheral De Novo Generation of Tregs

(a) In vivo conversion of naïve CD4+CD25-T cells expressing a TCR of interest into Tregs into Foxp3 expressing Treg using a RAG deficient host. The antigen is administered to a RAG deficient mouse carrying the transgenic TCR of interest (e.g., a mouse that carries only T cells which express the TCR for the insulin B9:23 peptide). The mouse can be 4-6 weeks old. Antigen presentation is carried out under subimmunogenic conditions, such subcutaneously using peptide infusion by an osmotic pump continuously over 14 days or injected as a covalent conjugate of the mimetope and an antibody that targets dendritic cells, such as an anti-DEC205 antibody. Analysis of Foxp3− expression in CD4+ enriched T cells from spleen and lymph nodes is carried out via FACS after 14 days.

(b) In vivo conversion of naïve CD4+CD25-T cells expressing a transgenic TCR of interest into Treg cells using a congenic transfer system. HA(107-119)TCR transgenic CD4+/Foxp3-/GFP Thy1.1+ cells are transferred iv into a host such as a Balb/c thy1.2+ mouse. A to single dose of a covalent conjugate of DEC205-HA(107-199) fusion antibody is administered ip. Analysis of Foxp3 expression in CD4-enriched Thy1.1+ spleenocytes and lymph nodes is conducted after 14 days flow cytometrically.

### 3. Testing of Mimetopes for Induction of Tregs

The inventors have been successful in inducing HY-specific Treg from naIve T cells in wt female B6 mice that were infused with HY-peptide and visualized with HY-peptide, class II tetramers (1). However, a similar approach to prevent diabetes in NOD mice by injecting insulin B:9-23 peptide into young NOD mice failed.

a. Insulin Mimetopes

The inventors performed similar protocols of antigen-specific Treg conversion studies to prevent the development of spontaneous diabetes in NOD mice by injecting DEC205 fusion antibodies that contain the beta chain of insulin. While these experiments showed a delay in the onset of disease, these studies were not able to prevent disease. Compelling data suggest that the Insulin B:9-23 and the IAg⁷ molecule form a loose and flexible complex. These results pointed to a direct relationship between a weak-peptide MHC and the development of an autoimmune T cell repertoire. The inventors hypothesized that weak epitopes may fail to induce efficient Treg conversion, and we initiated studies using Insulin B:9-23 TCR transgenic mice and identified in T cell proliferation assays insulin mimetopes that stimulate better than the Insulin B:9-23 peptide itself because of an increased binding capacity to MHC class II IAg⁷, compared to the aforementioned Insulin B:9-23 peptide. In fact such mimetopes achieved better conversion of naive T cells expressing the transgenic TCR. The inventors then studied the impact of these insulin-derived mimetopes in young NOD mice on diabetes development via subimmunogenic peptide delivery using either newly designed DEC205 peptide fusion antibodies (see description below) or peptide-infusing mini-osmotic pumps. In order to make the antigen specific Treg induction more efficient the inventors also optimized protocols using a combination of compounds that favor conversion of naive T cells into Tregs.

b. Enhanced Treg Conversion In Vivo and In Vitro

For the exploitation of antigen-specific Treg induction to interfere with unwanted immunity as diabetes the inventors performed optimization studies in order to achieve stable de novo differentiation of naive T cells into Tregs in sufficient numbers. Therefore we aimed at using combinations of compounds that enhance conversion, selectively expand the generated Tregs and allow for maintenance of the Treg phenotype. Recent data showed that the de novo expression of Foxp3 can be induced when TCR signaling is terminated prematurely, or by pharmacological inhibition of the mTOR/Akt pathway in activated T cells. Using a congenic adoptive-transfer system of HA(107-119) TCR transgenic Foxp3GFP-reporter Balbc mice the inventors performed in vivo experiments demonstrating that injection of the mTOR-inhibitor Everolimus substantially increases the frequency of newly generated HA-specific Foxp3+ Tregs. To induce rapid expansion of these Tregs the inventors injected the mice with IL-2 mixed with an optimized amount of a particular IL-2 monoclonal antibody (IL-2/IL-2 ab complexes) for a short period of 3 days at the end of the conversion period. By time kinetic analysis using the aforementioned congenic HA-TCR transgenic Foxp3-GFP-reporter system the inventors could show that mTOR-inhibition during conversion combined with IL-2/IL-2 ab complexes generated a significantly increased number of antigen-specific Tregs compared to HA-specific conversion in the absence of drugs. Moreover, these Tregs were stable and persisted over a period of 4 months, which is an important feature as it allows for prospective induction to suppress unwanted immune responses.

c. Novel DEC205 Peptide Fusion Antibodies

In order to facilitate the production of anti-DEC205-peptide fusion antibodies and to allow straightforward installation of peptides covalently linked to the DEC205-antibody, the inventors adopted the use of sortase-mediated transpeptidation or sortagging, a versatile orthogonal protein labeling method. Initiated by a sorting signal, LPETG, bacterial sortases covalently attach proteins to the bacterial cell wall. Synthetic peptides containing 2-5 glycines at the N-terminus readily serve as nucleophiles and allow the coupling of any peptide of interest. In collaboration with Hidde Ploegh (Whitehead Institute, MIT, Cambridge, Mass.) constructs encoding the anti-DEC205 Ig heavy chain with the LPETG tag have been used. Newly generated sortagged-DEC205 antibodies containing the above described Insulin mimetopes have been tested for antigen processing and presentation in vitro using T cells from Insulin B:9-23 TCR transgenic to mice and will now be used for antigen-specific Treg induction studies in vivo in order to exploit their potential to prevent diabetes.

d. Role of Retinoic Acid (RA) in Treg Conversion

Retinoic acid produced by specialized dendritic cells in the gut was shown to enhance antigen-specific Treg conversion in the presence of TGFb. Initial studies on the possible mechanisms concluded that RA counteracted the inhibitory effect of cytokines such as IL-4 or IL-21 on conversion when generated by activated T cells. The inventors' results yielded additional mechanisms that were not related to secreted inhibitory cytokines: RA could directly counteract the negative effect of costimulation on conversion by naive T cells in the absence of secreted cytokines. Since RA could also increase Smad3 expression an analysis was warranted whether this represented an important pathway of enhancing TGFβ-dependent Treg conversion. The results showed that RA could strongly enhance Treg conversion in Smad3- and Smad4-deficient mice by a RA receptor dependent mechanism that is likely to directly upregulate Foxp3 expression.

The results show that Treg conversion can be considerably enhanced by choice of appropriate mimetopes as well as drugs that make Treg conversion more efficient. With these novel tools the inventors then took aim at prevention of type 1 diabetes in NOD mice by the conversion of naive T cells with specificity for insulin into insulin-specific Treg cells. The inventors used various protocols of antigen-delivery (osmotic pumps, DEC205 sortagged antibodies) to deliver mimetopes for epitopes recognized by diabetes inducing T cell clones. The inventors produced tetramers of class II MHC molecules containing mimetope peptides recognized by disease causing T cell clones in order to monitor insulin-specific T cells during and after the conversion process as well as to isolate such Treg and using them in adoptive transfer systems. The overall goal was to induce such Treg in vivo in order to prevent the spontaneous development of disease.

e. The Results are Shown in the Figures.

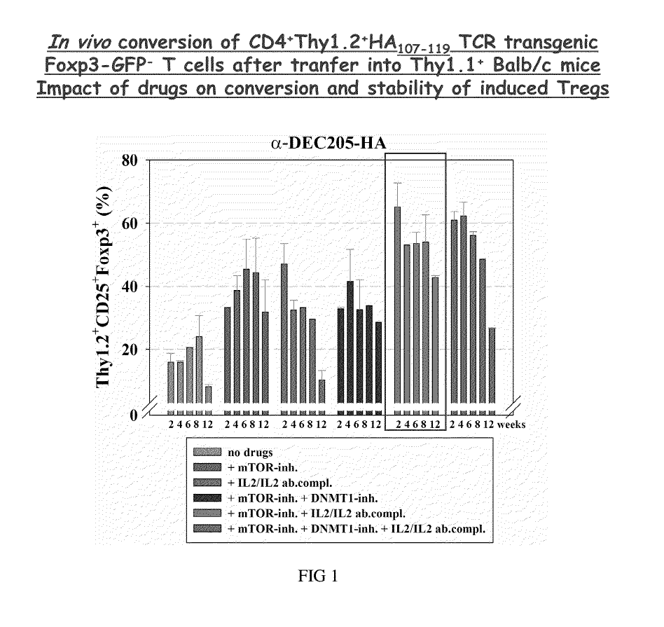

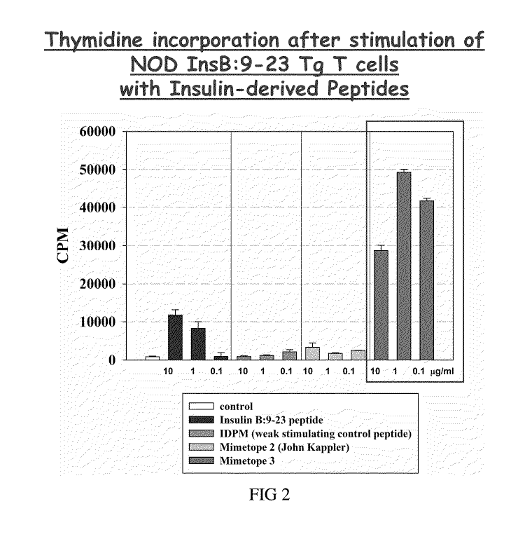

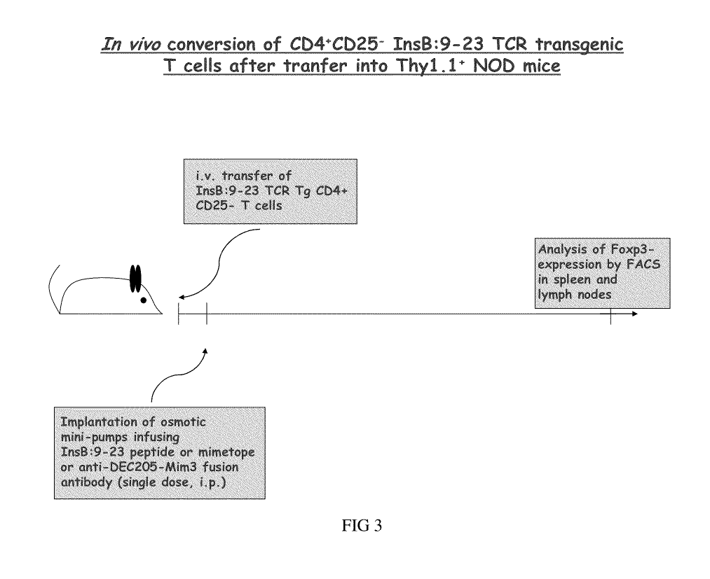

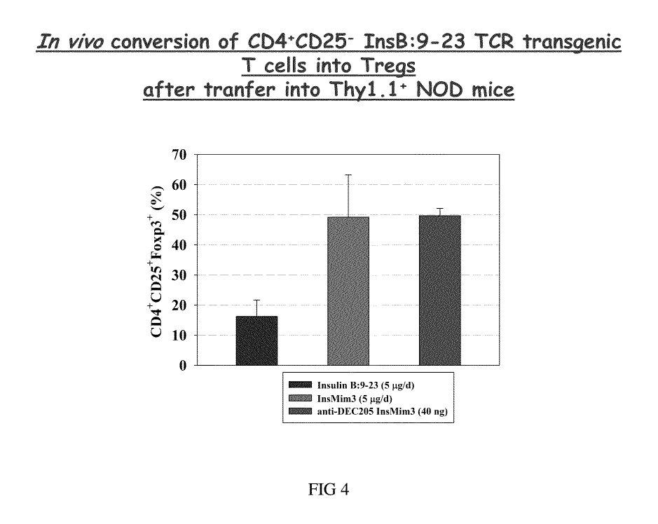

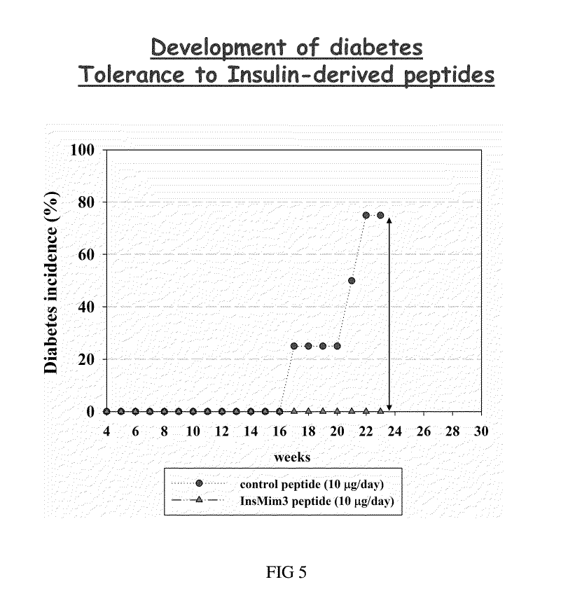

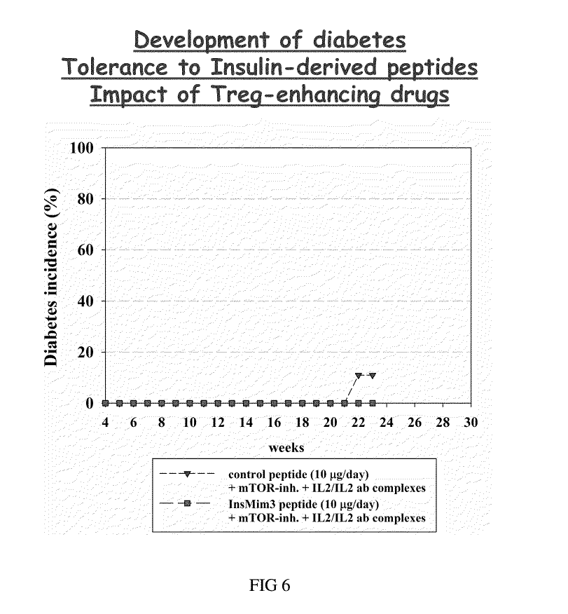

FIG. 1 shows the approach to make the antigen-specific conversion of naïve T cells into regulatory T cells more efficient by the use of certain drugs that enhance Treg conversion as to well as increase their stability. Time kinetic of Foxp3 expression at 2 week intervals is shown. FIG. 2 shows thymidine incorporation after stimulation of NOD InsB:9-23 Tg T cells with the natural Insulin B9:23 peptide or with identified Insulin mimetopes to identify better stimulating agonists. FIG. 3 shows the experimental scheme for the antigen-specific in vivo conversion protocol using an adoptive transfer system of naïve CD4+CD25− InsB:9-23 TCR transgenic T cells into congenic NOD mice. Antigen is supplied under subimmunogenic condition either in the form of peptide infusing osmotic pumps or by the use of DEC205 fusion antibodies targeting dendritic cells. FIG. 4 shows percentages of CD4+CD25+Foxp3+ Tregs after antigen-dependent in vivo conversion of naïve CD4+CD25− InsB:9-23 TCR transgenic T cells into Tregs by the use of an adoptive transfer system into congenic NOD hosts. FIG. 5 shows the diabetes incidence of normal female NOD mice that have received prospective Treg vaccination in form of subimmunogenic delivery of either the natural insulin B 9:23 peptide or the relevant insulin mimetope at the age of 4 weeks. FIG. 6 shows the impact of Treg-enhancing drugs on the diabetes incidence of normal female NOD mice that have received prospective Treg vaccination in form of subimmunogenic delivery of either the natural insulin B 9:23 peptide or the relevant insulin mimetope accompanied by a 2 weeks treatment scheme with Treg-enhancing drugs starting at the age of 4 weeks.

### REFERENCES

1. Klein L, Khazaie K, von Boehmer H (2003) In vivo dynamics of antigen-specific regulatory T cells not predicted from behavior in vitro. Proc Natl Acad Sci USA 100:8886-8891.
2. Mempel T R, et al. (2006) Regulatory T cells reversibly suppress cytotoxic T cell function independent of effector differentiation. Immunity 25:129-141.
3. Kim J M, Rasmussen J P, Rudensky A Y (2007) Regulatory T cells prevent catastrophic autoimmunity throughout the lifespan of mice. Nat Immunol 8:191-197.
4. Wise M P, Bemelman F, Cobbold S P, Waldmann H (1998) Linked suppression of skin graft rejection can operate through indirect recognition. J Immunol 161:5813-5816.
5. Karim M, Feng G, Wood K J, Bushell A R (2005) CD25⁺CD4⁺ regulatory T cells generated by exposure to a model protein antigen prevent allograft rejection: Antigen-specific reactivation in vivo is critical for bystander regulation. Blood 105:4871-4877.
6. Hoffman P, Ermann J, Edinger M, Fathman C G, Strober S (2002) Donor-type CD4⁺CD25⁺ regulatory T cells suppress lethal acute graft-vs-host disease after alogeneic bone to marrow transplantation. J Exp Med 196:389-399.
7. Trenado A, et al. (2003) Recipient-type specific CD4⁺C25⁺ regulatory T cells favor immune reconstitution and control graft-vs-host disease while maintaining graft-vs-leukemia. J Clin Invest 112:1688-1696.
8. Jaeckel E, von Boehmer H, Manns M P (2005) Antigen-specific FoxP3-transduced T-cells can control established type 1 diabetes. Diabetes 54:306-310.
9. Tang Q, et al. (2004) In vitro-expanded antigen-specific regulatory T cells suppress autoimmune diabetes. J Exp Med 199:1455-1465.
10. Read S, Malmstrom V, Powrie F (2000) Cytotoxic T lymphocyte-associated antigen 4 plays an essential role in the function of CD25⁺CD4⁺ regulatory cells that control intestinal inflammation. J Exp Med 192:295-302
11. Edinger M, et al. (2003) CD4⁺C25⁺ regulatory T cells preserve graft-vs-tumor activity while inhibiting graft-vs-host disease after bone marrow transplantation. Nat Med 9:1144-1150.
12. Wood K J, Sakaguchi S (2003) Regulatory T cells in transplantation tolerance. Nat Rev Immunol 3:199-210.
13. Cobbold S P, et al. (2004) Induction of foxP3⁺ regulatory T cells in the periphery of T cell receptor transgenic mice tolerized to transplants. J Immunol 172:6003-6010.
14. Ochando J C, et al. (2006) Alloantigen-presenting plasmacytoid dendritic cells mediate tolerance to vascularized grafts. Nat Immunol 7:652-662.
15. Jordan M S, et al. (2001) Thymic selection of CD4⁺C25⁺ regulatory T cells induced by an agonist self-peptide. Nat Immunol 2:301-306.
16. Apostolou I, Sarukhan A, Klein L, von Boehmer H (2002) Origin of regulatory T cells with known specificity for antigen. Nat Immunol 3:756-763.
17. Apostolou I, von Boehmer H (2004) In vivo instruction of suppressor commitment in naïve T cells. J Exp Med 199:1401-1408.
18. Kretschmer K, et al. (2005) Inducing and expanding regulatory T cell populations by to foreign antigen, Nat Immunol 6:1219-1227.
19. Chen T C, Cobbold S P, Fairchild P J, Waldmann H (2004) Generation of anergic and regulatory T cells following prolonged exposure to a harmless antigen. J Immunol 172:5900-5907.
20. Mahnke K, Qian Y, Knop J, Enk A H (2003) Induction of CD4⁺C25⁺ regulatory T cells by targeting of antigens to immature dendritic cells. Blood 101:4862-4869.
21. Thorstenson K M, Khoruts A (2001) Generation of anergic and potentially immuno-regulatory C25⁺ CD4 T cells in vivo after induction of peripheral tolerance with intravenous or oral antigen. J Immunol 167:188-195.
22. Simpson E, et al. (2002) Minor H antigens: Genes and peptides. Transpl Immunol 10:115-123.
23. Millrain M, et al. (2001) Examination of HY response: T cell expansion, immunodominance, and cross-priming revealed by HY tetramer analysis. J Immunol 167:3756-3764.
24. Fontenot J D, et al. (2005) Regulatory T cell lineage specification by the forkhead transcription factor foxp3. Immunity 22:329-341.
25. Chai J G, James E, Dewchand H, Simpson E, Scott D (2004) Transplantation tolerance induced by intranasal administration of HY peptides. Blood 103:3951-3959.
26. Joffre O, van Meerwijk J P (2006) CD4⁺C25⁺ regulatory T lymphocytes in bone marrow transplantation. Semin Immunol 18:128-135.
27. Hanash A M, Levy R B (2005) Donor CD4⁺C25⁺ regulatory T cells promote engraftment and tolerance following MHC-mismatched hematopoietic cell transplantation. Blood 105:1828-1836.
28. Lantz O, Grandjean I, Matzinger P, Di Santo J P (2000) Gamma chain required for naïve CD4⁺ T cell survival but not for antigen proliferation. Nat Immunol 1:54-58.
29. Kisielow P, Bluthmann H, Staerz U D, Steinmetz M, von Boehmer H (1988) Tolerance in T-cell-receptor transgenic mice involves deletion of nonmature CD4⁺8⁺ thymocytes. Nature 333:742-746.
30. Virginis P, et al. (2008) Induction of antigen-specific regulatory T cells in wild-type mice: Visualization and targets of suppression. PNAS 105: 3479-3484.

What is claimed is:

1. A method for increasing in a subject Treg cells specific for an antigen, comprising
administering to the subject, under sub-immunogenic conditions, a mimetope of the antigen, wherein the mimetope has a mimetope TCP index, and wherein the mimetope TCP index is at least 25% greater than the antigen TCP index under the same conditions of T cell proliferation.

2. The method of claim 1, wherein the mimetope TCP index is greater, under the same conditions, as the antigen TCP index by at least 50%, 100%, 200%, 300%, 400%, 500% or 1000%.

3. The method of claim 1, wherein the mimetope is administered under sub-immunogenic conditions subcutaneously by an osmotic pump.

4. The method of claim 1, wherein the mimetope is administered under sub-immunogenic conditions by injection of a covalent conjugate of the mimetope and an antibody that binds specifically to dendritic cells.

5. The method of claim 1, wherein the mimetope is a peptide and the antigen is a native protein.

6. The method of claim 5, wherein the peptide is represented by a sequence of contiguous amino acids, which sequence is present as contiguous amino acids found in the protein, except for one amino acid, and wherein the one amino acid is part of an MHC binding portion of the mimetope.

7. The method of claim 5, wherein the peptide is represented by a sequence of contiguous amino acids, which sequence is present as contiguous amino acids found in the protein, except for two amino acids, and wherein at least one of the two amino acids is part of an MHC binding portion of the mimetope.

8. The method of claim 7, wherein each of the two amino acids is part of the MHC binding portion of the mimetope.

9. The method of claim 5, wherein the peptide comprises a sequence X₁, X₂, X₃, X₄, X₅, X₆, X₇, wherein each X represents an amino acid, and wherein the sequence differs from a sequence of contiguous amino acids found within the protein at only one of X₁, X₂, X₃, X₄, X₅, X₆, X₇, and wherein said only one is part of an MHC binding portion of the mimetope.

10. The method of claim 5, wherein the peptide comprises a sequence X₁, X₂, X₃, X₄, X₅, X₆, X₇, wherein each X represents an amino acid, and wherein the sequence differs from a sequence of contiguous amino acids found within the protein at only two of X₁, X₂, X₃, X₄, X₅, X₆, X₇, and wherein said only two are part of the same MHC binding portion of the mimetope.

11. The method of claim 5, wherein the antigen is a self antigen involved in autoimmune disease.

12. The method of claim 11, wherein the autoimmune disease is Multiple Sclerosis, autoimmune myocarditis, pemphigus, celiac disease, myasthenia gravis, Hashimoto's thyroiditis, Graves' disease, Addison's disease, chronic lyme arthritis, Goodpasture syndrome, Kawasaki disease, scleroderma, Sjogren's syndrome.

13. The method of claim 5, wherein the antigen is insulin, myelin basic protein, cardiac myosin, Pemphigus vulgaris antigen (Desmoglein 3), gliadin, muscle acetylcholine receptor, ryanodine receptor 1, thyroid peroxidase, thyroglobulin, thyroid peroxidase, a sodium-iodide symporter, a thyrotropin receptor, a cytoplasmic adrenal antigen, a P450 enzyme 21-hydroxylase, a 11 beta-hydroxylase, a 17 alpha-hydroxylase, a side-chain cleavage enzyme P450, a 3 beta-hydroxysteroid dehydrogenase, a polypeptide of Borrelia Burgdorferi and its outer surface proteins, a collagen, type IV, alpha 3, a factor VIII related antigen (von Willebrand's Factor), an adenoviral antigen, a protein subunit of human RNAse P, a fibrillarin, antigen, or a Lupus La protein.

14. The method of claim 5, wherein the mimetope is a mimetope of natural insulin B:9-23 peptide, Chromogranin A or myelin basic protein.

15. The method of claim 5, wherein the mimetope is InsMim3 peptide mimetope, InsMim8 peptide mimetope, pS3 peptide mimetope, Ac1-11 A4 MBP peptide mimetope, or Ac1-11 Y4 MBP peptide mimetope.

16. The method of claim 5, wherein the subject is a candidate for a transplant and the antigen is present in the transplant but not in the subject.

17. The method of claim 5, wherein the antigen is a MHC antigen.

18. The method of claim 15, wherein the antigen is a MHC antigen.

19. The method of claim 18, wherein the mimetope is E62M peptide mimetope.

20. The method of claim 5, wherein the antigen is an allergen.

21. The method of claim 1, wherein the subject does not have detectable cytotoxic T cells specific for the antigen.

22. A pharmaceutical preparation for inhibiting autoimmune disease comprising a sub-immunogenic dose of a peptide mimetope of a self antigen involved in auto-immune disease, wherein the peptide mimetope has a mimetope TCP index, and wherein the mimetope TCP index is at least 25% greater than an antigen TCP index under the same conditions of T cell proliferation.

23. The pharmaceutical preparation of claim 22, wherein the peptide mimetope has a sequence X₁, X₂, X₃, X₄, X₅, X₆, X₇, wherein each X represents an amino acid, and wherein the sequence differs from a sequence of contiguous amino acids found within the antigen at only one or two of X₁, X₂, X₃, X₄, X₅, X₆, X₇, and wherein said only one or two is part of an MHC binding portion of the peptide mimetope and the antigen.

24. The pharmaceutical preparation of claim 23, wherein the self antigen is involved in Multiple Sclerosis, autoimmune myocarditis, pemphigus, celiac disease, myasthenia gravis, Hashimoto's thyroiditis, Graves' disease, Addison's disease, chronic lyme arthritis, Goodpasture syndrome, Kawasaki disease, scleroderma, or Sjogren's syndrome.

25. The pharmaceutical preparation of claim 24, wherein the antigen is insulin, myelin basic protein, cardiac myosin, Pemphigus vulgaris antigen (Desmoglein 3), gliadin, muscle acetylcholine receptor, ryanodine receptor 1, thyroid peroxidase, thyroglobulin, thyroid peroxidase, a sodium-iodide symporter, a thyrotropin receptor, a cytoplasmic adrenal antigen, a P450 enzyme 21-hydroxylase, a 11 beta-hydroxylase, a 17 alpha-hydroxylase, a side-chain cleavage enzyme P450, a 3 beta-hydroxysteroid dehydrogenase, a polypeptide of Borrelia Burgdorferi and its outer surface proteins, a collagen, type IV, alpha 3, a factor VIII related antigen (von Willebrand's Factor), an adenoviral antigen, a protein subunit of human RNAse P, a fibrillarin, antigen, or a Lupus La protein.

26. The pharmaceutical preparation of claim 22 wherein the peptide mimetope is a mimetope of natural insulin B:9-23 peptide.

27. The pharmaceutical preparation of claim 22, wherein the pharmaceutical preparation is in an osmotic pump.

28. The pharmaceutical preparation of claim 22, wherein the mimetope is covalently conjugated to an antibody that binds specifically to a dendritic cell.

29. A pharmaceutical preparation for treating transplant rejection comprising a sub-immunogenic dose of a peptide mimetope of a non-self transplant antigen, wherein the peptide mimetope has a mimetope TCP index, and wherein the mimetope TCP index is at least 25% greater than an antigen TCP index under the same conditions of T cell proliferation.

30. The pharmaceutical preparation of claim 29, wherein the peptide mimetope has a sequence X₁, X₂, X₃, X₄, X₅, X₆, X₇, wherein each X represents an amino acid, and wherein the sequence differs from a sequence of contiguous amino acids found within the antigen at only one or two of X₁, X₂, X₃, X₄, X₅, X₆, X₇, and wherein said only one or two is part of an MHC binding portion of the peptide mimetope and the antigen.

31. The pharmaceutical preparation of claim 29, wherein the antigen is an MHC antigen.

32. The pharmaceutical preparation of claim 29, wherein the pharmaceutical preparation is in an osmotic pump.

33. The pharmaceutical preparation of claim 29, wherein the mimetope is covalently conjugated to an antibody that binds specifically to a dendritic cell.


SEQUENCE LISTING (10 sequences)

SEQ ID NO: 1 - protein, 15 aa, Artificial sequence
  Insulin B peptide
SHLVEALYLVCGERG

SEQ ID NO: 2 - protein, 12 aa, Artificial sequence
  InsMim3 peptide mimetope
VEALYLVCGEEG

SEQ ID NO: 3 - protein, 12 aa, Artificial sequence
  InsMim8 peptide mimetope
VEALYLVAGEEG

SEQ ID NO: 4 - protein, 14 aa, Artificial sequence
  WE14 peptide
WSRMDQLAKELTAE

SEQ ID NO: 5 - protein, 9 aa, Artificial sequence
  pS3 peptide mimetope
RLGLWVRME

SEQ ID NO: 6 - protein, 15 aa, Artificial sequence
  Kd 54-68 peptide
QEGPEYWEEQTQRAK

SEQ ID NO: 7 - protein, 15 aa, Artificial sequence
  E62M peptide mimetope
QEGPEYWEMQTQRAK

SEQ ID NO: 8 - protein, 11 aa, Artificial sequence
  1-11 MBP peptide
ASQKRPSQRHG

SEQ ID NO: 9 - protein, 11 aa, Artificial sequence
  1-11 A4 MBP peptide mimetope
ASQARPSQRHG

SEQ ID NO: 10 - protein, 11 aa, Artificial sequence
  1-11 Y4 MBP peptide mimetope
ASQYRPSQRHG